(19) United States
(12) Patent Application Publication
(10) Pub. No.: US 2024/0059973 A1
(43) Pub. Date: Feb. 22, 2024

(54) CAPPED ALKOXYLATED ALCOHOLS

(71) Applicant: Arkema France, COLOMBES (FR)

(72) Inventors: Jean-Philippe Gillet, Pierre-Benite (FR); Carl Bouret, Chateauroux (FR); Tony Bartolini, Colombes (FR)

(73) Assignee: Arkema France, Colombes (FR)

(21) Appl. No.: 17/766,647

(22) PCT Filed: Oct. 16, 2020
(86) PCT No.: PCT/FR2020/051856

§ 371 Date: Apr. 5, 2022

(30) Foreign Application Priority Data
Oct. 18, 2019 (FR) 1911676

(51) Int. Cl. C09K 23/38

(52) U.S. Cl. CPC C09K 23/38

(57) ABSTRACT

The invention relates to a composition comprising a mixture of C₃-C₂₂ alcohol alkoxylates which have a narrow weight distribution and are capped in the terminal portion by a group chosen from linear or branched alkyls comprising between 1 and 6 carbon atoms, the phenyl group, benzyl group and hydrocarbon groups having a carboxy function —COO—, and groups having a sugar unit. The invention also relates to the method for preparing said composition and to the uses thereof as a surfactant, in particular as a surfactant with low foaming power.

### CROSS-REFERENCE TO RELATED APPLICATIONS

This application is the U.S. national phase of International Application No. PCT/FR2020/051856, filed 16 Oct. 2020, which claims priority to French Application No. FR 1911676, filed 18 Oct. 2019, the disclosure of each of these applications being incorporated herein by reference in its entirety for all purposes.

### FIELD OF THE INVENTION

The present invention relates to the general field of alkoxylated alcohols and more particularly to capped alkoxylated alcohols, to their process of preparation and also to their uses as surface-active agents.

### BACKGROUND OF THE INVENTION

It is now known that alcohol alkoxylates represent a family of compounds offering a wide range of properties, with multiple applications, such as solvents, hydrotropic agents or surface-active agents. Thus, alcohol alkoxylates constitute a class of compounds exhibiting real industrial advantage for a great many fields of application.

Conventionally, alcohol alkoxylates are synthesized with the help of basic catalysis, using for example potassium hydroxide, referred to as “potassium hydroxide catalysis” or also “KOH catalysis”. For about ten years, however, another type of catalyst has been presented as being able to be used under certain conditions with certain reactants in order to obtain alkoxylates. This is the catalyst of the double metal cyanide type, also called DMC catalyst.

Already in the 1960s, the patent U.S. Pat. No. 3,359,331 dealt with the ethoxylation of alcohols using a catalyst based on tin and antimony. The catalyst was used in a relatively large amount, in a reaction medium at a temperature of less than 70° C. and at a pressure close to atmospheric pressure. As this type of catalyst is very fragile, it was impossible to work in conventional reactors at the risk of deactivating the catalyst.

Many years later, researchers of repute published studies (di Serio M. et al., Ind. Eng. Chem. Res., (1996) 35, 3848-3853) relating to the comparative kinetics of the ethoxylation and propoxylation of 1- and 2-octanol by KOH catalysis. The authors concluded that KOH catalysis is not satisfactory and encouraged the development of more efficient catalysts.

More recently, the international application WO2009000852 described a process for the alkoxylation of various compounds having mobile H, including alcohols, by DMC catalysis. This document teaches the need to add an oxypropylene (OP) and/or oxybutylene (OB) block to the starting substrate, before being able to graft an oxyethylene (OE) block, by DMC catalysis. The great majority of the substrates are alcohols of Neodol type (polybranched alcohols obtained by the Fischer-Tropsch process) and of primary type. 97In addition, the catalyst concentrations employed are high, approximately 3% by weight with respect to the starting product.

Similarly, the international application WO2012005897 discloses the alkoxylation of alcohols by DMC catalysis, comprising first of all the addition of OP blocks and only subsequently the addition of OE blocks.

The absence of large amounts of alcohol alkoxylates on the market currently suggests that DMC catalysis seems today difficult to implement industrially, in particular on alcohol-type substrates, whereas this type of catalysis might make it possible to obtain alkoxylates with entirely noteworthy properties, in particular alcohol alkoxylates capped in the terminal position (or end-capped).

Some end-capped alkoxylates have already been described, such as, for example, those with benzyl termination in the patent EP 2205711 or those with carboxylic termination described in the international application WO2004037960.

It is well known that alkoxylation reactions lead to mixtures of alkoxylated products comprising a variable number of alkoxyl groups, the number of alkoxyl units in said mixture of alkoxylated products most often following a more or less broad or more or less narrow Gaussian distribution, generally characterized by the width of the Gaussian curve at mid-height, commonly quantified statistically by the 2a value.

It has now been discovered, entirely surprisingly, that it is possible to prepare, in a particularly easy way industrially, end-capped alcohol alkoxylates which exhibit entirely advantageous properties, in terms of physicochemical properties as well as in terms of application properties.

### SUMMARY OF THE INVENTION

Thus and according to a first aspect, the present invention relates to a composition comprising a mixture of end-capped alcohol alkoxylates, in which composition:
the alcohol comprises from 3 to 22, preferably from 5 to 22, carbon atoms, more preferably from 5 to 20, very particularly preferably from 5 to 18, carbon atoms,
the weight distribution of the alkoxylates follows a monomodal distribution, the peak width value (2a) of which is less than 7, preferably less than 6, advantageously less than 5, more preferably less than 4, and
the end part is capped by a group chosen from linear or branched alkyls comprising from 1 to 6 carbon atoms, the phenyl group, the benzyl group, the hydrocarbon groups carrying a carboxy —COO— functional group, and the groups carrying a sugar unit.

Preferably, the end cap of the alcohol alkoxylates is chosen from methyl, ethyl, propyl, butyl and benzyl groups and alkylcarboxyl —COOH group and its salts. Mention may be made, among the salts of the carboxyl functional group which can be envisaged, of the salts well known to a person skilled in the art and in particular the metal, alkali metal, alkaline earth metal or ammonium salts, to mention only the main among them. The sodium, potassium, calcium and ammonium salts are very particularly preferred salts.

According to another embodiment, the end cap of the alcohol alkoxylates is chosen from alkylenecarboxyls and its salts, which are optionally functionalized. A typical and nonlimiting example is represented by the sulfosuccinate group and in particular sodium, potassium, calcium and ammonium sulfosuccinates

According to yet another embodiment, the end cap of the alcohol alkoxylates is chosen from groups carrying one sugar unit, such as, for example, glucose (case of monoglucosides), or two or more sugar units (case of alkylpolyglucosides, also referred to as “APGs”).

As indicated above, the alcohol used as starting substrate for the alkoxylation reaction(s) comprises from 3 to 22, preferably from 5 to 22, carbon atoms, more preferably from 5 to 20, very particularly preferably from 5 to 18, carbon atoms. The carbon atoms can be in a linear, branched or partially or completely cyclic chain. According to a preferred embodiment, the alcohol has a weight-average molar mass ranging from 45 g·mol⁻¹ to 300 g·mol⁻¹, preferably from 70 g·mol⁻¹ to 250 g·mol⁻¹ and more preferably from 80 g·mol⁻¹ to 200 g·mol⁻¹.

### DETAILED DESCRIPTION OF THE INVENTION

The alcohol used as starting substrate can be of any type and any origin. Generally, the alcohol is a primary alcohol or a secondary alcohol. It can be of petroleum origin or of biobased origin, for example of plant or animal origin. An alcohol of biobased origin is preferred, for obvious reasons of environmental protection. It is also preferred to use a secondary alcohol for the requirements of the present invention.

When the alcohol is a primary alcohol, it can be chosen from linear or branched primary alcohols, for example from linear or branched primary alcohols comprising from 8 to 14 carbon atoms, for example 1-octanol, 1-nonanol, 1-decanol, 1-undecanol, 1-dodecanol, 1-tridecanol or 1-tetradecanol, in particular alcohols having 10 carbon atoms, such as Exxal™ 10, or also alcohols having 13 carbon atoms, such as Exxal™ 13, which are sold, for example, by Exxon Mobil.

When the alcohol is a secondary alcohol, it can be chosen from linear or branched secondary alcohols comprising from 3 to 22 carbon atoms and optionally comprising one or more aromatic group(s), the representatives of which can be phenolic alcohols, such as, for example, cardanol. According to a very particularly preferred aspect, the secondary alcohol comprises from 3 to 22 carbon atoms, entirely advantageously from 3 to 14 carbon atoms, more preferably from 6 to 12 carbon atoms. More preferably, the secondary alcohol is chosen from 2-octanol and 4-methyl-2-pentanol; very particularly preferably, the secondary alcohol is 2-octanol.

The alkoxylated repeat units are chosen from ethylene oxide, propylene oxide and butylene oxide units and their mixtures.

Within the meaning of the present invention, the term “ethylene oxide unit” is understood to mean a unit resulting from ethylene oxide after opening of the oxirane ring. Within the meaning of the present invention, the term “propylene oxide unit” is understood to mean a unit resulting from propylene oxide after opening of the oxirane ring. Within the meaning of the present invention, the term “butylene oxide unit” is understood to mean a unit resulting from butylene oxide after opening of the oxirane ring.

According to one embodiment of the present invention, the capped alcohol alkoxylates comprise a sequence comprising one or more units chosen from the ethylene oxide unit, the propylene oxide unit, the butylene oxide unit and their mixtures, said units being distributed randomly, alternately or in blocks.

According to another embodiment of the present invention, the capped alcohol alkoxylates comprise ethylene oxide units and a sequence comprising one or more units chosen from the ethylene oxide unit, the propylene oxide unit, the butylene oxide unit and their mixtures, it being possible for said units to be distributed randomly, alternately or in blocks, at least one propylene oxide or butylene oxide unit being present in said sequence.

According to another preferred embodiment, the capped alcohol alkoxylates comprise at least one ethylene oxide unit and at least one propylene oxide unit, distributed alternately, randomly or in blocks.

Still according to yet another preferred embodiment, the capped alcohol alkoxylates comprise at least one ethylene oxide unit and at least one butylene oxide unit, distributed alternately, randomly or in blocks.

Another embodiment of the invention relates to the capped alcohol alkoxylates comprising at least one propylene oxide unit and at least one butylene oxide unit, distributed alternately, randomly or in blocks.

The number of repeat units is generally between, limits included, 1 and 100, preferably between 2 and 100, more preferably between 3 and 100, particularly between 3 and 80, more particularly between 3 and 75, preferably between 3 and 50, limits included.

According to a preferred embodiment of the present invention, the number of repeat units is between, limits included, 1 and 75, preferably between 2 and 75, more preferably between 3 and 75, particularly between 4 and 75, more particularly between 5 and 75, preferably between 6 and 75, more preferably between 7 and 75, preferably between 8 and 75, more preferred between 9 and 75 and very preferably between 10 and 75.

According to another preferred embodiment, the number of repeat units is between, limits included, 1 and 50, preferably between 2 and 50, more preferably between 3 and 50, particularly between 4 and 50, more particularly between 5 and 50, preferably between 6 and 50, more preferably between 7 and 50, preferably between 8 and 50, more preferred between 9 and 50 and very preferably between 10 and 50.

According to yet another preferred embodiment, the number of repeat units is between, limits included, 1 and 30, preferably between 2 and 20, more preferably between 3 and 20 and advantageously between 3 and 15.

In the composition of the invention, the capped alcohol alkoxylates are present according to a monomodal weight distribution according to a normal law of statistical distribution. According to a very specific aspect of the present invention, the composition of secondary alcohol alkoxylates exhibits a narrow monomodal weight distribution.

In the present description and claims, the weight distribution is determined by analysis by gas chromatography on a standard column and flame ionization detection (FID) well known to a person skilled in the art, where the various components of the compositions analyzed are separated by increasing boiling point and thus by increasing molar mass by addition each time of an alkylene oxide unit. The weight distributions correspond to surface area percentages regarded as equivalent to percentages by weight, on the assumption that the products have the same response coefficient, since they are of the same chemical nature.

It has been discovered, entirely surprisingly, that this very particularly narrow monomodal distribution of the capped alcohol alkoxylates present in the composition according to the present invention can be obtained using an alkoxylation reaction in presence of a specific catalyst making possible very good control of the alkoxylation reaction and in particular in the presence of a catalyst of double metal cyanide (DMC) type. Other known catalysts making possible access to mixtures of alkoxylates having a narrow range distribution can also be used and, as such, mention may be made, in a nonlimiting way, of acid catalysis of BF₃ derivatives type, calcium-based basic catalysis, hydrotalcite-type catalysts, and others. However, for the requirements of the present invention, catalysts of DMC type, as indicated above, are preferred.

This is because it has been able to be observed that, in the presence of such a specific “narrow range” catalyst, the weight distribution of the alkoxylates is narrow and very particularly narrower than with basic catalysis of potassium hydroxide catalysis type.

In addition to obtaining compositions having a very broad weight distribution, it is known that the reactions for the alkoxylation of substrate, in particular when the substrate is an alcohol and very particularly when the alcohol is a secondary alcohol, by the conventional routes (basic catalysis) leads to a very significant residual amount of unreacted substrate.

The capping reaction carried out on such compositions having a broad distribution and significant residual amount can present difficulties of implementation (reaction media which can be viscous, making then problematic to handle, insufficient yields, and others) and thus lead, in certain cases, to capped alkoxylate compositions with application properties which are not very acceptable, indeed even mediocre. It is moreover very probably this which explains why, until now, such capped alkoxylates have not been developed industrially at the present time.

On the other hand, and this is one of the very particular advantages of the present invention, the capped alcohol alkoxylates and very particularly the capped secondary alcohol alkoxylates described here exhibit a tight distribution and, quite unexpectedly, greatly improved application performance qualities. In particular, when the compositions according to the present invention are used as surface-active agents, a reduced foaming effect and better detergent performance qualities can be observed, compared with the compositions known and available on the market today.

It is also possible to obtain the compositions according to the present invention by carrying out the capping reaction described above directly on narrow range alkoxylates already commercially available. Mention may be made, among these narrow range alkoxylates, for example, of those of the Berol® range, sold by Nouryon.

Some of the capped alcohol alkoxylates described in the present disclosure are novel and, as such, come within the scope of the present invention.

Thus, and according to another aspect, the invention relates to a composition comprising a mixture of capped 2-octanol alkoxylates having a narrow weight distribution, with a peak width value (2σ) of less than 7, preferably of less than 6, more preferably of less than 5, entirely preferably of less than 4.

More specifically, the invention relates to a composition comprising 2-octanol alkoxylates capped by a group chosen from linear or branched alkyls comprising from 1 to 6 carbon atoms, the phenyl group, the benzyl group, the hydrocarbon groups carrying a carboxy —COO— functional group, and the groups carrying a sugar unit, as defined above.

More specifically still, the present invention relates to a composition comprising:
2-octanol which is ethoxylated and then capped with propylene oxide,
2-octanol which is ethoxylated and then capped with butylene oxide,
2-octanol which is ethoxylated and/or propoxylated and then capped by an alkyl group, in particular chosen from methyl, ethyl, propyl or butyl, or also by a benzyl group,
2-octanol which is ethoxylated and/or propoxylated and then capped by a carboxyl (—(CH₂)ₙ—COOH, where n is an integer between 1 and 5, limits included, optionally in the alkali metal, alkaline earth metal or ammonium, preferably Na⁺, K⁺ or NH₄⁺, salt form).

According to a very particularly preferred aspect, the present invention relates to a composition comprising:
2-octanol 2-15 OE 1 OP,
benzyl-capped 2-octanol 2-15 OE,
methyl-capped 2-octanol 2-15 OE,
ethyl-capped 2-octanol 2-15 OE,
propyl-capped 2-octanol 2-15 OE,
butyl-capped 2-octanol 2-15 OE,
CH₂—COOH-capped 2-octanol 2-15 OE,
2-octanol 2-15 OE 1-15 OB,
2-octanol 2-15 OE 1-15 OP,
2-octanol 1-6 OE 1-15 OP.

Another subject matter of the present invention is a process for the preparation of the compositions according to the present invention as defined above, comprising the following successive stages:
a) reacting an alcohol with one or more alkylene oxides chosen from ethylene oxide, propylene oxide, butylene oxide and their mixtures, in the presence of at least one alkoxylation catalyst of narrow range type, preferably of DMC type;
b) reacting the product resulting from stage (a) with one or more compounds capable of carrying out an end capping.

The alkoxylation of stage a) can be carried out with one or more alkylene oxides, simultaneously, sequentially or alternately, depending on the order of the alkoxylated units which are desired in the final composition.

The alkylene oxides employed in the process of the present invention can be of diverse origins, and in particular “mass balance” alkylene oxides, especially “mass balance” ethylene oxide, alkylene oxides of biobased origin. Advantageously, the ethylene oxide used is of biobased origin; for example, the ethylene oxide can be obtained by oxidation of biobased ethylene originating from the dehydration of bioethanol, itself originating from corn starch, from lignocellulose materials, from agricultural waste, such as, for example, sugar cane bagasse, and others.

As indicated above, the alkoxylation reaction is carried out in the presence of a catalyst resulting in a narrow weight distribution of the alkoxylates obtained and preferably with the lowest possible residual amount of alcohol. An entirely suitable catalyst belongs to the family of the catalysts of double metal cyanide (DMC) type.

Optionally, the product resulting from stage (a) can be isolated, although this is not necessary, in particular because the residual content of starting alcohol is quite minimal and negligible.

The alcohol employed in stage a) of the process of the invention can be any alcohol known to a person skilled in the art and in particular is as described above; the alcohol is chosen from primary and secondary alcohols, preferably from secondary alcohols and preferably from 2-octanol and methyl isobutyl carbinol, the preferred alcohol being 2-octanol.

This is because 2-octanol exhibits a very particular advantage in several respects, in particular because it results from a biobased product which does not compete with human or animal food. Furthermore, 2-octanol, which has a high boiling point, is biodegradable and exhibits a good ecotoxicological profile.

According to a preferred embodiment, the alcohol is employed in stage a) after drying, according to conventional techniques well known to a person skilled in the art, so that the water content in said secondary alcohol is less than or equal to 200 ppm, preferably less than or equal to 100 ppm.

Preferably, the catalyst which can be used for the alkoxylation reaction of stage a) of the process of the present invention can be any narrow range catalyst known to a person skilled in the art and in particular a catalyst of double metal cyanide (DMC) type. When the catalyst is of double metal cyanide type, it can be of any nature well known to a person skilled in the art and as described, for example, in the patents U.S. Pat. Nos. 6,429,342, 6,977,236 and PL398518. More particularly, the catalyst used comprises zinc hexacyanocobaltate and one or more ligands, for example the catalyst sold by Covestro under the name Arcol® or the catalyst sold by Mexeo under the name MEO-DMC®.

Advantageously, the content of catalyst of double metal cyanide type ranges from 1 ppm to 1000 ppm, with respect to the content of starting alcohol, preferably from 1 ppm to 500 ppm, preferably from 2 ppm to 300 ppm, more preferably from 5 ppm to 200 ppm.

The reaction can be carried out under all temperature and pressure conditions, as is well known to a person skilled in the art, and, according to a preferred embodiment, the reaction temperature during the alkoxylation stage (a) is generally between 80° C. and 200° C., preferably between 100° C. and 180° C. The reaction pressure during stage (a) can range from 0.01 MPa to 3 MPa, preferably from 0.02 MPa to 2 MPa.

Preferably, the process according to the invention comprises a stage of removal of the residual oxides used in the alkoxylation and/or capping stage, more particularly the ethylene oxide, the propylene oxide, the butylene oxide and their mixtures employed during the process according to the invention. Thus, this stage can take place after stage (a) and/or after stage (b), preferably after stage a).

Within the meaning of the present invention, the term “residual oxide” is understood to mean an oxide which has not reacted. Preferably, said stage of removal of the residual oxide is carried out by cooking, that is to say by maintaining a temperature ranging from 70° C. to 170° C., preferably from 100° C. to 160° C., in order to consume the residual oxide, and/or by a stage of stripping under a stream of inert gas. Alternatively, said stripping stage can be carried out under reduced pressure.

Preferably, after said removal stage, the content by weight of residual oxide is generally less than or equal to 0.05%, with respect to the total weight of alkoxylates, capped or not, depending on whether this removal stage is carried out before or after stage b), preferably less than or equal to 0.01%, more preferably less than or equal to 0.001%.

The end capping or capping reaction (stage b) is carried out in a conventional way, according to any method known to a person skilled in the art, with or without a catalyst, and as, for example, described in the documents EP 2 205 711 and WO2004037960, cited supra. In general, this capping reaction is carried out after formation of the alkoxide, in a basic medium (KOH or NaOH, for example), or else in the presence of a catalyst of narrow range type, as described above, and especially a catalyst of DMC type, in particular when the capping is carried out using an alkylene oxide. Typically, the alkoxylate or the mixtures of alkoxylates is/are reacted in the alkoxide form with a halide (for example alkyl halide, benzyl halide, ω-halogenated carboxylic acid halide, and others) or else with an alkylene oxide. The reaction medium is subsequently neutralized, the salt formed is filtered off and the expected product is recovered. When it is chosen to carry out the capping reaction in the presence of a catalyst of narrow range type and in particular a catalyst of DMC type, it can be advantageous to use the same catalyst as that used in stage a), indeed even without proceeding to a fresh addition of catalyst, and to use the catalyst which was used during stage a).

The process according to the present invention can be carried out batchwise, semicontinuously or continuously. A person skilled in the art will know how to adapt the process for the manufacture of the compositions according to the invention according to the distribution, random, alternating or in blocks, of the sequences of alkoxylates desired.

In addition, the process according to the invention exhibits the advantage of synthesizing the capped alcohol alkoxylates under good safety conditions, so that it can be carried out on an industrial scale. This is because the operating conditions in terms of temperature and of pressure are controlled by virtue of the process according to the invention. In particular, the exothermicity of the reaction can be controlled very easily.

The compositions of capped alcohol alkoxylates can most often be used as is, on leaving the reactor, without it being necessary to provide other stages of purification, distillation or others. If necessary, conventional operations of filtration, drying, purification, and others, can be carried out.

Finally, a subject matter of the present invention is the use of a composition of capped alcohol alkoxylates according to the present invention as surface-active agent and in particular as surface-active agent having a low foaming power (low-foaming surfactant).

This is because the compositions of the present invention, which are characterized in particular by a narrow weight distribution, exhibit very advantageous application properties in terms of performance. Furthermore, the compositions of the present invention exhibit entirely advantageous biodegradability profiles, in particular for low levels of alkoxylation (<8 units).

The capped alcohol alkoxylates having a narrow weight distribution make them compositions entirely suitable in a very large number of fields of application, such as, for example and in a nonlimiting way, for detergents, for cosmetic products, for the flotation of ores, as lubricant, in particular for metal working fluids, as emulsifier, as adjuvant for bituminous applications, as wetting agent, as solvent, as coalescence agent, as processing aid, for deinking, as gas hydrate antiagglomerant, in enhanced gas and oil recovery applications, in corrosion protection, in hydraulic fracturing, in soil bioremediation, in agrochemicals (for example, coating of granular products, in particular fertilizers and plant protection products), but also as hydrotropic agent, antistatic agent, paint adjuvant, textile adjuvant, for polyols, for the production of electrodes and electrolytes for batteries, to mention only the main fields of application.

Another subject matter of the present invention is a formulation comprising at least one composition of capped alcohol alkoxylates as defined above and one or more aqueous, organic or aqueous/organic solvents chosen from water, alcohols, glycols, polyols, mineral oils, vegetable oils, waxes and others, alone or as mixtures of two or more of them, in all proportions.

The formulation according to the invention can also contain one or more additives and fillers well known to a person skilled in the art, such as, for example and in a nonlimiting way, anionic, cationic, amphoteric or nonionic surfactants, rheology modifiers, de-emulsifiers, deposition-inhibiting agents, antifoams, dispersants, pH control agents, colorants, antioxidants, preservatives, corrosion inhibitors, biocides and other additives, such as, for example, sulfur, boron, nitrogen or phosphorus products, and others. The natures and amounts of the additives and fillers can vary within wide proportions depending on the nature of the envisaged application and can easily be adjusted by a person skilled in the art.

The invention is now illustrated by the following examples which are in no way limiting.

### EXAMPLES

The 2-octanol (CAS RN 123-96-6) used is the “refined” grade 2-octanol Oleris® (purity>99%), sold by Arkema France.

### Example A: Comparison Between KOH Catalysis and DMC Catalysis

To illustrate the narrow distribution effect obtained by DMC catalysis, in comparison with a basic potassium hydroxide catalysis, a test of alkoxylation of 2-octanol, in a proportion of 1 mol of 2-octanol per 2 mol of propylene oxide, is carried out under the same operating conditions, on the one hand with a KOH catalyst and on the other hand with a DMC catalyst.

In both cases, the 2-octanol is dried beforehand (to less than 1000 ppm for KOH and less than 200 ppm for DMC). The amount of catalyst is equal to 2500 ppm of KOH, on the one hand, and to 100 ppm of DMC, on the other hand. The reaction is carried out in an autoclave under pressure of between 0.15 MPa and 0.6 MPa, at a temperature of between 130° C. and 170° C. The results, in terms of weight distribution of the alkoxylation compounds, determined by gas chromatography and expressed as % of surface area of peaks of each of the alkoxylates, are presented in the following table 1:

TABLE 1
Weight distribution 2-octanol 2 OP

| No. OP | 0 | 1 | 2 | 3 | 4 | 5 | 6 | 7 | 8 | 9 | 10 |
|---|---|---|---|---|---|---|---|---|---|---|---|
| KOH | 28 | 16 | 14 | 10 | 6 | 5 | 4 | 3 | 2 | 2 | 1 |
| DMC | 8 | 18 | 26 | 24 | 14 | 5 | 2 | 1 | 0 | 0 | 0 |

It is found, with this example, that, in DMC catalysis, the distribution is overall centered on a number of OP units equal to 2. It is also noted that the residual amount of alcohol (No. OP=0) is markedly lower in the case of DMC catalysis than in the case of KOH catalysis.

Furthermore, the 2a value calculated with the values resulting from the basic catalysis is 5.0, whereas this 2a value calculated with the values resulting from the DMC catalysis is 2.9.

### Example 1: Synthesis of 2-Octanol 6 OE 4 OP in DMC Catalysis

750 g (5.76 mol) of 2-octanol, dried to less than 200 ppm of water, and 0.11 g (150 ppm) of DMC catalyst Arcol® are charged to a clean and dry 4 l autoclave. The reactor is closed and purged with nitrogen and the leaktightness under pressure is checked. The reactor is pressurized with nitrogen. The reaction medium is to start with brought to 90° C. with stirring. 30 g of ethylene oxide are introduced at the temperature of 120° C. When initiation of the reaction is observed, the balance of the ethylene oxide, i.e. in total 1520 g (34.56 mol), is introduced over a period of time of 2 h 50 min at a temperature of approximately 140° C. At the end of the addition, the temperature is maintained for 30 min and then the residual ethylene oxide is stripped off with nitrogen. The reactor is cooled to 80° C. and 1000 g of expected product are withdrawn: 2-octanol 6 OE (I_{OH}: 138 mg KOH/g and coloration of 77 Hz).

20 g of propylene oxide are introduced at a temperature of 130° C. onto the 1270 g (3.22 mol) of 2-octanol 6 OE remaining in the reactor. When initiation of the reaction is observed, the balance of the propylene oxide, i.e. in total 747 g (12.9 mol), is introduced over a period of time of 55 min at a temperature of approximately 140° C. At the end of the addition, the temperature is maintained for 30 min and then the residual propylene oxide is stripped off with nitrogen.

At the end of the reaction, 2015 g of clear 2-octanol 6 OE 4 OP are recovered at 50° C. (I_{OH}: 86 mg KOH/g and coloration of 10 Hz).

### Example 2: Synthesis of 2-Octanol 6 OE 4 OB by DMC Catalysis

500 g (3.84 mol) of 2-octanol, dried to less than 200 ppm of water, and 0.075 g (150 ppm) of DMC catalyst Arcol® are charged to a clean and dry 4 l autoclave. The reactor is closed and purged with nitrogen and the leaktightness under pressure is checked. The reactor is pressurized with nitrogen. The reaction medium is to start with brought to 90° C. with stirring. 25 g of ethylene oxide are introduced at the temperature of 120° C. When initiation of the reaction is observed, the balance of the ethylene oxide, i.e. in total 1015 g (23 mol), is introduced over a period of time of 2 h at a temperature of approximately 140° C. At the end of the addition, the temperature is maintained for 30 min and then the residual ethylene oxide is stripped off with nitrogen. The reactor is cooled to 80° C. and 1000 g of product are withdrawn: 2-octanol 6 OE (I_{OH}: 140 mg KOH/g and coloration of 50 Hz). 20 g of butylene oxide are introduced at a temperature of 130° C. onto the 513 g (1.3 mol) of 2-octanol 6 OE remaining in the reactor. When initiation of the reaction is observed, the balance of the butylene oxide, i.e. in total 375 g (5.2 mol), is introduced over a period of time of 45 min at a temperature of approximately 140° C. At the end of the addition, the temperature is maintained for 30 min and then the residual butylene oxide is stripped off with nitrogen.

At the end of the reaction, 880 g of clear 2-octanol 6 OE 4 OB are recovered at 50° C. (I_{OH}: 81 mg KOH/g and coloration of 20 Hz).

### Example 3: Synthesis of 2-Octanol 13 OE Benzyl Ether in DMC Catalysis

500 g (3.84 mol) of 2-octanol, dried to less than 200 ppm of water, and 0.075 g (150 ppm) of DMC catalyst Arcol® are charged to a clean and dry 4 l autoclave. The reactor is closed and purged with nitrogen and the leaktightness under pressure is checked. The reactor is pressurized with nitrogen. The reaction medium is to start with brought to 90° C. with stirring. 30 g of ethylene oxide are introduced at the temperature of 120° C. When initiation of the reaction is observed, the balance of the ethylene oxide, i.e. in total 2200 g (50 mol), is introduced over a period of time of 3 h at a temperature of approximately 140° C. At the end of the addition, the temperature is maintained for 30 min and then the residual ethylene oxide is stripped off with nitrogen. The reactor is cooled to 80° C. and 2700 g of product are withdrawn: 2-octanol 13 OE (I_{OH}: 78 mg KOH/g and coloration of 20 Hz). The product is a white solid at ambient temperature.

2106 g (3 mol) of 2-octanol 13 OE obtained above and also 10 g of water are charged to a 4 l glass reactor provided with a mechanical stirrer, with heating, with a dropping funnel for introduction of solid and with a system for rendering inert with nitrogen. The reaction medium is brought to 90° C. while sparging with nitrogen in order to deoxygenate the medium. Nitrogen is subsequently placed in the headspace of the reactor and then 132 g (3.3 mol) of sodium hydroxide beads, i.e. 15% excess, are added. The medium is subsequently brought to 100° C.-105° C. and under pressure reduced to approximately 300 mbar, so as to distil off the water. The halting criterion is a water content<1.5%. The reaction medium is subsequently brought back to 70° C. and then 342 g (2.7 mol) of benzyl chloride are added over approximately 60 min. The temperature is maintained at 120° C. for 5 h. After returning to 70° C., the reaction medium is neutralized with 37% hydrochloric acid until a pH of 7 is obtained. The water is distilled off under reduced pressure in order to precipitate the sodium chloride formed. The latter is filtered off and 2300 g of benzyl-capped 2-octanol 13 OE are recovered.

### Example 4: Synthesis of 2-Octanol 9 OE Ether Carboxylic in DMC Catalysis

500 g (3.84 mol) of 2-octanol, dried to less than 200 ppm of water, and 0.075 g (150 ppm) of DMC catalyst Arcol® are charged to a clean and dry 4 l autoclave. The reactor is closed and purged with nitrogen and the leaktightness under pressure is checked. The reactor is pressurized with nitrogen. The reaction medium is to start with brought to 90° C. with stirring. 25 g of ethylene oxide are introduced at the temperature of 120° C. When initiation of the reaction is observed, the balance of the ethylene oxide, i.e. in total 1520 g (34.56 mol), is introduced over a period of time of 2 h 30 at a temperature of approximately 140° C. At the end of the addition, the temperature is maintained for 30 min and then the residual ethylene oxide is stripped off with nitrogen. The reactor is cooled to 80° C. and 2010 g of product are withdrawn: 2-octanol 9 OE (I_{OH}: 105 mg KOH/g and coloration of 35 Hz).

1578 g (3 mol) of 2-octanol 9 OE obtained above are charged to a 3 l glass reactor provided with a mechanical stirrer, with heating, with a dropping funnel for introduction of solid and with a system for rendering inert with nitrogen. The reaction medium is brought to 50° C. while sparging with nitrogen in order to deoxygenate the medium. Nitrogen is subsequently placed in the headspace of the reactor and then 126 g (3.15 mol) of sodium hydroxide beads are added. The water is distilled off under reduced pressure. 367 g (3.15 mol) of sodium monochloroacetate are then added at 50° C. At the end of the reaction, the reaction medium is neutralized with 37% hydrochloric acid. 1610 g of 2-octanol 9 OE ether carboxylic are recovered.

### Example 5: Synthesis of 1-Decanol 5 OE by Basic KOH Catalysis

500 g (3.16 mol) of 1-decanol of biobased origin (sold by Ecogreen), dried to less than 1000 ppm of water, and 1.5 g (3000 ppm) of potassium hydroxide (KOH) catalyst as pellets are charged to a clean and dry 4 l autoclave. The reactor is closed and purged with nitrogen and the leaktightness under pressure is checked. The reactor is pressurized with nitrogen. The reaction medium is to start with brought to 90° C. with stirring. 30 g of ethylene oxide are introduced at the temperature of 120° C. When initiation of the reaction is observed, the balance of the ethylene oxide, i.e. in total 695 g (15.8 mol), is introduced over 1 hour at a temperature of approximately 140° C. At the end of the addition, the temperature is maintained for 30 min and then the residual ethylene oxide is stripped off with nitrogen. The reactor is cooled to 80° C. and the withdrawal is carried out of 1180 g of crude product, 1-decanol 5 OE, which is neutralized with acetic acid (I_{OH}: 153 mg KOH/g and coloration of 385 Hz).

### Example 6: Synthesis of 1-Decanol 5 OE by DMC Catalysis

500 g (3.16 mol) of 1-decanol of biobased origin (sold by Ecogreen), dried to less than 200 ppm of water, and 0.075 g (150 ppm) of DMC catalyst (sold by Mexeo) are charged to a clean and dry 4 l autoclave. The reactor is closed and purged with nitrogen and the leaktightness under pressure is checked. The reactor is pressurized with nitrogen. The reaction medium is to start with brought to 90° C. with stirring. 35 g of ethylene oxide are introduced at the temperature of 120° C. When initiation of the reaction is observed, the balance of the ethylene oxide, i.e. in total 695 g (15.8 mol), is introduced over 1 hour at a temperature of approximately 140° C. At the end of the addition, the temperature is maintained for 30 min and then the residual ethylene oxide is stripped off with nitrogen. The reactor is cooled to 80° C. and 1185 g of product are withdrawn: 1-decanol 5 OE (I_{OH}: 145 mg KOH/g and coloration of 23 Hz).

The results, in terms of weight distribution of the alkoxylation compounds, determined by gas chromatography and expressed as % of surface area of peaks of each of the alkoxylates, are presented in the following table 2:

TABLE 2
Weight distribution 1-decanol 5 OE

| No. OE | 0 | 1 | 2 | 3 | 4 | 5 | 6 | 7 |
|---|---|---|---|---|---|---|---|---|
| KOH | 9.82 | 7.17 | 8.06 | 8.78 | 8.92 | 8.21 | 8.03 | 7.7 |
| DMC | 1.2 | 1.77 | 4.29 | 10.33 | 18.44 | 22.74 | 19.52 | 11.88 |
| No. OE | 8 | 9 | 10 | 11 | 12 | 13 | 14 | 15 |
| KOH | 7.11 | 5.85 | 4.7 | 3.72 | 2.61 | 1.77 | 1.18 | 0 |
| DMC | 5.52 | 2.03 | 0.72 | 0.25 | 0.08 | 0.03 | 0 | 0 |

The 2a value calculated with the values resulting from the basic catalysis is 7.3, whereas this 2a value calculated with the values resulting from the DMC catalysis is 3.7.

### Example 7: Synthesis of 1-Decanol 13 OE, Benzylated, Potassium Hydroxide (KOH) Catalysis

Stage a): Ethoxylation

500 g (3.16 mol) of biobased 1-decanol (sold by Ecogreen), dried to less than 1000 ppm of water, and 1.5 g (3000 ppm) of solid KOH are charged to a clean and dry 4 l autoclave. The reactor is closed and purged with nitrogen and the leaktightness under pressure is checked. The reactor is pressurized with nitrogen. The reaction medium is to start with brought to 90° C. with stirring. 30 g of ethylene oxide are introduced at the temperature of 120° C. When initiation of the reaction is observed, the balance of the ethylene oxide, i.e. in total 1807 g (41 mol), is introduced over 2 hours 40 min at a temperature of approximately 140° C. At the end of the addition, the temperature is maintained for 30 min and then the residual ethylene oxide is stripped off with nitrogen. The reactor is cooled to 80° C. and 2281 g of product are withdrawn: 1-decanol 13 OE (I_{OH}: 77 mg KOH/g and coloration of 480 Hz on the molten product). The product is a white solid at ambient temperature.

Stage b): Capping

2000 g (2.74 mol) of 1-decanol 13 OE obtained in the preceding stage and also 10 g of water are charged to a 4 l glass reactor provided with a mechanical stirrer, with heating, with a dropping funnel for introduction of solid and with a system for rendering inert with nitrogen. The reaction medium is brought to 90° C. while sparging with nitrogen in order to deoxygenate the medium. Nitrogen is subsequently placed in the headspace of the reactor and then 120 g (3 mol) of sodium hydroxide beads are added. The medium is subsequently brought to 100° C.-105° C. and under pressure reduced to approximately 30 kPa, so as to distil off the water. The halting criterion is a water content of less than 1.5%. The reaction medium is subsequently brought back to 70° C. 329 g (2.6 mol) of benzyl chloride are then added over approximately 60 min. The temperature is maintained at 120° C. for 5 hours. After returning to 70° C., the reaction medium is neutralized with 37% hydrochloric acid until a pH of 7 is obtained. The water is distilled off under reduced pressure in order to precipitate the sodium chloride formed. The latter is filtered off and 2195 g of benzyl-capped 1-decanol 13 OE are recovered.

### Example 8: Synthesis of 1-Decanol 13 OE, Benzylated, DMC Catalysis

Stage a): Ethoxylation

500 g (3.16 mol) of biosourced 1-decanol, dried to less than 200 ppm of water, and 0.075 g (150 ppm) of DMC catalyst Arcol® are charged to a clean and dry 4 l autoclave. The reactor is closed and purged with nitrogen and the leaktightness under pressure is checked. The reactor is pressurized with nitrogen. The reaction medium is to start with brought to 90° C. with stirring. 35 g of ethylene oxide are introduced at the temperature of 120° C. When initiation of the reaction is observed, the balance of the ethylene oxide, i.e. in total 1807 g (41 mol), is introduced over 2 hours 40 min at a temperature of approximately 140° C. At the end of the addition, the temperature is maintained for 30 min and then the residual ethylene oxide is stripped off with nitrogen. The reactor is cooled to 80° C. and 2290 g of product are withdrawn: 1-decanol 13 OE (I_{OH}: 75 mg KOH/g and coloration of 30 Hz on the molten product). The product is a white solid at ambient temperature.

Stage b): Capping

2190 g (3 mol) of 1-decanol 13 OE obtained above and also 10 g of water are charged to a 4 l glass reactor provided with a mechanical stirrer, with heating, with a dropping funnel for introduction of solid and with a system for rendering inert with nitrogen. The reaction medium is brought to 90° C. while sparging with nitrogen in order to deoxygenate the medium. Nitrogen is subsequently placed in the headspace of the reactor and then 132 g (3.3 mol) of sodium hydroxide beads are added. The medium is subsequently brought to 100° C.-105° C. and under pressure reduced to approximately 30 kPa, so as to distil off the water. The halting criterion is a water content of less than 1.5%. The reaction medium is subsequently brought back to 70° C. and 366 g (2.9 mol) of benzyl chloride are added over approximately 60 min. The temperature is maintained at 120° C. for 5 hours. After returning to 70° C., the reaction medium is neutralized with 37% hydrochloric acid until a pH of 7 is obtained. The water is distilled off under reduced pressure in order to precipitate the sodium chloride formed. The latter is filtered off and 2390 g of benzyl-capped 1-decanol 13 OE are recovered.

### Example 9: Synthesis of 2-Octanol 3 OE Disodium Sulfosuccinate Monoester

393 g (1.5 mol) of 2-octanol 3 OE, prepared by means of a DMC catalyst as described in WO2019092366, are charged to a 2 l glass reactor provided with a stirrer and a system for the introduction of solids.

The reaction medium is brought to a temperature of between 60° C. and 70° C. and then 154 g (1.57 mol) of maleic anhydride are gradually introduced with stirring while maintaining the temperature. After addition, the temperature is maintained at 70° C. for one hour. The degree of esterification is then checked by quantitative determination. 816 g of a 20% aqueous sodium bisulfite solution (i.e. 1.57 mol) are subsequently run in, with stirring, at a temperature of between 75° C. and 90° C. After addition, the reaction medium is maintained at 90° C. When the reaction is complete, the reaction medium is cooled, the pH is adjusted by addition of a sodium hydroxide solution and the reactor is emptied.

### Example 10: Synthesis of 2 Octanol 3 OE Monoglucoside

655 g (2.5 mol) of 2-octanol 3 OE, prepared by means of DMC catalysis as described in WO2019092366, 90 g (0.5 mol) of glucose and 7.45 g of para-toluenesulfonic acid, i.e. 1% of the reaction medium, are charged to a 1 l glass reactor provided with a stirrer, a dropping funnel, an electric heating system and a system for placing under reduced pressure.

The medium is brought to 115° C. with stirring and under an inert atmosphere. The assembly is then gradually placed under pressure reduced down to a value of 30 mmHg (i.e. 4 kPa). The water formed is distilled off and collected in a cold trap. The reaction is continued for approximately 7 hours, so as to convert all of the glucose.

Cooling is carried out and the catalyst is neutralized with sodium hydroxide. The excess ethoxylated alcohol can be recovered by distillation under reduced pressure using the WFSP (Wiped Film Short Path) technology.

1. A composition comprising a mixture of end-capped alcohol alkoxylates, wherein:
the alcohol comprises from 3 to 22 carbon atoms,
the weight distribution of the alkoxylates follows a monomodal distribution, the peak width value (2σ) of which is less than 7, and
an end part is capped by a group chosen from linear or branched alkyls comprising from 1 to 6 carbon atoms, a phenyl group, a benzyl group, hydrocarbon groups carrying a carboxy —COO— functional group, and groups carrying a sugar unit.

2. The composition as claimed in claim 1, wherein the end cap of the alcohol alkoxylates is chosen from methyl, ethyl, propyl, butyl or benzyl groups, carboxyl —COOH groups and its salts, and alkylenecarboxyl groups and its salts, which are optionally functionalized, including sulfosuccinate group.

3. The composition as claimed in claim 1, wherein the alcohol is a primary alcohol or a secondary alcohol.

4. The composition as claimed in claim 3, wherein the alcohol is a primary alcohol chosen from linear or branched primary alcohols comprising from 8 to 14 carbon atoms.

5. The composition as claimed in claim 3, wherein the alcohol is a linear or branched secondary alcohol comprising from 3 to 22 carbon atoms, optionally comprising one or more aromatic group(s).

6. The composition as claimed in claim 3, wherein the alcohol is a secondary alcohol comprising from 3 to 14 carbon atoms.

7. The composition as claimed in claim 1, wherein the capped alcohol alkoxylates comprise a sequence comprising one or more units chosen from an ethylene oxide unit, a propylene oxide unit, a butylene oxide unit and their mixtures, the units being distributed randomly, alternately or in blocks.

8. The composition as claimed in claim 1, comprising a mixture of 2-octanol alkoxylates capped by a group chosen from linear or branched alkyls comprising from 1 to 6 carbon atoms, a phenyl group, a benzyl group, hydrocarbon groups carrying a carboxy —COO— functional group, and groups carrying a sugar unit.

9. The composition as claimed in claim 1, comprising:
2-octanol which is ethoxylated and then capped with propylene oxide,
2-octanol which is ethoxylated and then capped with butylene oxide,
2-octanol which is ethoxylated and/or propoxylated and then capped by an alkyl group, or also by a benzyl group,
2-octanol which is ethoxylated and/or propoxylated and then capped by a carboxyl (—(CH₂)ₙ—COOH, where n is an integer between 1 and 5, limits included, optionally in an alkali metal, alkaline earth metal or ammonium salt form).

10. The composition as claimed in claim 1, comprising:
2-octanol 2-15 OE 1 OP,
benzyl-capped 2-octanol 2-15 OE,
methyl-capped 2-octanol 2-15 OE,
ethyl-capped 2-octanol 2-15 OE,
propyl-capped 2-octanol 2-15 OE,
butyl-capped 2-octanol 2-15 OE,
CH₂—COOH-capped 2-octanol 2-15 OE,
2-octanol 2-15 OE 1-15 OB,
2-octanol 2-15 OE 1-15 OP,
2-octanol 1-6 OE 1-15 OP.

11. A process for the preparation of a composition as claimed in claim 1, comprising the following successive stages:
a) reacting an alcohol with one or more alkylene oxides chosen from ethylene oxide, propylene oxide, butylene oxide and their mixtures, in the presence of at least one alkoxylation catalyst of narrow range type;
b) reacting the product resulting from stage (a) with one or more compounds capable of carrying out an end capping.

12. The use of a composition as claimed in claim 1 as a surface-active agent.

13. The use as claimed in claim 12, for detergents, for cosmetic products, for the flotation of ores, as lubricant, as emulsifier, as adjuvant for bituminous applications, as wetting agent, as solvent, as coalescence agent, as processing aid, as gas hydrate antiagglomerant, for deinking, in enhanced gas and oil recovery applications, in corrosion protection, in hydraulic fracturing, in soil bioremediation, in agrochemicals, as hydrotropic agent, antistatic agent, paint adjuvant, textile adjuvant, for polyols, for the production of electrodes and electrolytes for batteries.

14. A formulation comprising at least one composition as claimed in claim 1 and one or more aqueous, organic or aqueous/organic solvents chosen from water, alcohols, glycols, polyols, mineral oils, vegetable oils, waxes and others, alone or as mixtures of two or more of them, in all proportions.
